(19) United States
(12) Patent Application Publication
(10) Pub. No.: US 2026/0011420 A1
(43) Pub. Date: Jan. 8, 2026

(54) Health Dashboard

(71) Applicants: Melina Aria Malkani, Bethesda, MD (US); Dillon Arvin Malkani, Bethesda, MD (US); Tara Neville Bamji, Scarsdale, NY (US)

(72) Inventors: Melina Aria Malkani, Bethesda, MD (US); Dillon Arvin Malkani, Bethesda, MD (US); Tara Neville Bamji, Scarsdale, NY (US)

(21) Appl. No.: 18/761,978

(22) Filed: Jul. 2, 2024

(51) Int. Cl. G16H 15/00; G16H 50/20

(52) U.S. Cl. CPC G16H 15/00; G06F 3/048; G16H 50/20

(57) ABSTRACT

The health dashboard of the present invention underscores the significance of healthcare dashboards in enabling the public to make informed healthcare decisions. This health dashboard is characterized by its user-friendly navigation, high usability, adjustable thresholds, and a variety of chart options. It complies with the Americans with Disabilities Act, includes charts with tabulated data, integrates user feedback, and features a simple design with clear chart descriptions and comparative data analysis. This invention allows the public to better comprehend healthcare information, enables providers to efficiently create dashboards without extensive resources, and incorporates AI and predictive analytics. Additionally, the dashboard is designed for compatibility with various devices, ensuring that users receive the appropriate amount of data for immediate healthcare decisions. Regardless of age or education level, the public can effectively understand and utilize the presented healthcare data, ultimately enhancing public healthcare outcomes.

### STATEMENT REGARDING PRIOR DISCLOSURES BY THE INVENTOR OR JOINT INVENTOR

Some primary ideas of this patent application were presented at a conference on Jul. 25, 2023.

### BACKGROUND OF THE INVENTION

### Technical Field

This invention relates generally to a health dashboard. This invention relates more particularly to a dashboard for effectively displaying health information such a COVID-19 reporting.

This invention relates generally to health dashboard for displaying information about health and disease. This invention relates more particularly to a health dashboard that has easy navigation, adjustable thresholds, diverse chart selection, compliance with the Americans with Disabilities Act, charts with tabulated data, user feedback incorporation, simplicity of design, clear chart descriptions, and comparative data analysis.

### Background Art

In the prior art, there are documents that focus on a distinct area, ranging from healthcare dashboard best practices to innovative patent applications in healthcare and economic analysis systems. Below are some specifics about the prior art available today:

There is a patent application for a web-based dashboard system designed for multi-scale, multi-regional visual, and spatial economic analysis with integrated business outreach. It discusses an interactive data visualization dashboard that helps users identify companies of interest within a super-region economy.

Another patent document is a distributed cloud computing system that using metadata and network data, the system derives metrics that span across the involved cloud networks. These metrics relate to the performance and characteristics of the gateways and the constructs associated with them. The system then generates a dashboard visualization that presents these metrics, providing a graphical representation of the system's status and performance across the networks. Finally, the dashboard is displayed on a screen, allowing users to visually monitor and assess the functioning and health of the cloud networks and their components.

Also in the prior art is a patent application that discusses a blockchain and cloud-based healthcare management system designed to provide enhanced healthcare management by utilizing blockchain technology to manage and analyze healthcare information.

The prior art also contains an interactive data visualization dashboard that provides detailed insights into a super-region's economy, enabling users to identify companies of interest. It features several linked and interactive charts: a main region chart showing major areas, a sub-region chart for detailed local areas, an industry sector chart for exploring industry specifics, a financial stress chart displaying economic stability, and a material change chart indicating significant regional changes. User interactions with one chart prompt corresponding updates across others, allowing iterative refinement and detailed analysis of the data displayed.

There are various COVID-19 healthcare dashboards that cover aspects such as reported deaths, vaccine coverage, and hospital resource use related to COVID-19. Further, there are dashboards for COVID-19 in specific U.S. states including county-level data, wastewater data, test positivity rates, and more. It discusses changes in reporting and the impact of these changes on understanding the pandemic's progress. Lastly, the World Health Organization (WHO) has a COVID-19 dashboard that offers a snapshot of the global status of the COVID-19 pandemic as reported by the WHO, including total cases, deaths, and vaccination doses administered.

In light of the foregoing prior art, there is a need for a health dashboard that has easy navigation, adjustable thresholds, diverse chart selection, compliance with the Americans with Disabilities Act, charts with tabulated data, user feedback incorporation, simplicity of design, clear chart descriptions, and comparative data analysis that is easier for a user to view a health information record of said user, a health condition of said user, and/or a disease condition of said user. This, therefore, improves said user's knowledge and said user's understanding of their health and/or condition.

The current invention is significantly more than just software; it represents a major leap forward in how health information is conveyed to users. This advanced system includes a specialized device and method for displaying elements that enhance a user's understanding of their health condition. The breakthrough is achieved through a careful combination and specific type of displayed images, meticulously chosen to effectively communicate vital health information. A standout feature of this invention is the consistent presentation of data, maintained by a uniform y-axis across all elements. This uniformity eliminates confusion, enabling users to easily compare different data points and gain a clearer, more accurate understanding of their health status. Beyond mere software, this invention incorporates advanced visualization techniques that highlight critical health indicators and trends, making it simpler for users to recognize patterns and potential issues. By embedding these sophisticated visual elements into health-related applications, the invention not only enhances user experience but also empowers individuals to make more informed decisions about their health and well-being. In essence, this innovative solution transcends traditional software by providing an integrated tool that revolutionizes how users interact with and interpret their health data. It sets a new benchmark for healthcare applications, offering users an unprecedented level of clarity and insight into their health.

### BRIEF SUMMARY OF THE INVENTION

The present invention highlights the importance of healthcare dashboards in helping the public make informed healthcare decisions. The health dashboard of the present invention encompasses easy navigation, high usability, the use of adjustable thresholds, diverse chart selection, compliance with the Americans with Disabilities Act, charts with tabulated data, user feedback incorporation, simplicity of design, clear chart descriptions, and comparative data analysis.

According to a first aspect of the invention, there is a user health dashboard for collecting and displaying a health information record of a user, a health condition of said user, and/or a disease condition having at least fifteen attributes of said user comprising a processor having a memory running an algorithm configured to present to said user a display window configured to show fifteen attributes of said health information record in up to six tiles having a common y-axis for a data display on a panel wherein said tiles are selected from a first data visualization configured to display at least one attribute of said health information record, a second data visualization configured to display at least one attribute of said health information record, a third data visualization configured to display at least one attribute of said health information record, a first bar chart configured to display at least one attribute of said health information record, a second bar chart configured to display at least one attribute of said health information record, and a historical chart configured to display at least one attribute of said health information record wherein all tiles use a common data element of an attribute such as a collection of entries or readings over time.

According to a second aspect of the invention, there is a user health dashboard wherein at least one tile comprises a prediction of a future condition of a display attribute.

According to a third aspect of the invention, there is a user health dashboard wherein said prediction of said future condition is derived from said algorithm and/or an artificial intelligence algorithm.

According to a fourth aspect of the invention, there is a user health dashboard having not less than three tiles consisting of said first data visualization, said first bar chart, and said historical chart.

According to a fifth aspect of the invention, there is a user health dashboard further configured for said health condition comprising a display of tiles for attributes related to said health condition.

According to a sixth aspect of the invention, there is a user health dashboard further configured for said disease condition comprising a display of tiles for attributes related to said disease condition.

According to a seventh aspect of the invention, there is a user health dashboard for collecting and displaying a health condition of a user having at least fifteen attributes of said user comprising a processor having a memory running an algorithm configured to present to said user a display window configured to show fifteen attributes of said health condition in up to six tiles having a common y-axis for a data display on a panel wherein said tiles are selected from a first data visualization configured to display at least one attribute of said health condition, a second data visualization configured to display at least one attribute of said health condition, a third data visualization configured to display at least one attribute of said health condition, a first bar chart configured to display at least one attribute of said health condition, a second bar chart configured to display at least one attribute of said health condition, and a historical chart configured to display at least one attribute of said health condition wherein all tiles use a common data element of an attribute such as a collection of entries or readings over time.

According to an eighth aspect of the invention, there is a user health dashboard wherein a tile comprises a prediction of a future condition of a display attribute.

According to a ninth aspect of the invention, there is a user health dashboard wherein said prediction of said future condition is derived from said algorithm and/or an artificial intelligence algorithm.

According to a tenth aspect of the invention, there is a user health dashboard having not less than three tiles consisting of said first data visualization, said first bar chart, and said historical chart.

According to an eleventh aspect of the invention, there is a user health dashboard for collecting and displaying a disease condition of a user having at least fifteen attributes of said user comprising a processor having a memory running an algorithm configured to present to said user a display window configured to show fifteen attributes of said disease condition in up to six tiles having a y-axis for a data display on a panel wherein said tiles are selected from a first data visualization configured to display at least one attribute of said disease condition, a second data visualization configured to display at least one attribute of said disease condition, a third data visualization configured to display at least one attribute of said disease condition, a first bar chart configured to display at least one attribute of said disease condition, a second bar chart configured to display at least one attribute of said disease condition, and a historical chart configured to display at least one attribute of said disease condition wherein all tiles use a common data element of an attribute such as a collection of entries or readings over time.

According to a twelfth aspect of the invention, there is a user health dashboard wherein a tile comprises a prediction of a future condition of a display attribute.

According to a thirteenth aspect of the invention, there is a user health dashboard wherein said prediction of said future condition is derived from said algorithm and/or an artificial intelligence algorithm.

According to a fourteenth aspect of the invention, there is a user health dashboard having not less than three tiles consisting of said first data visualization, said first bar chart, and said historical chart.

Advantages of the health dashboard are that it has easy navigation, adjustable thresholds, diverse chart selection, compliance with the Americans with Disabilities Act, charts with tabulated data, user feedback incorporation, simplicity of design, clear chart descriptions, and comparative data analysis. Further, the health dashboard of the present invention makes creating a health dashboard easier, more efficient, and more well-rounded. The health dashboard is also much easier to use from a public user point off view as the information is displayed in a more intuitive and easy to understand way.

The invention will now be described, by way of example only, with reference to the accompanying drawings in which:

### BRIEF DESCRIPTION OF THE SEVERAL VIEWS OF THE DRAWINGS

FIG. 1 is an example of a dashboard screen that displays data on the average length of stay, admissions, 30-day readmission rates, age group distributions, and treatment costs of the health dashboard according to the invention;

FIG. 2 is an example of a dashboard screen that provides information on nurse-patient ratios, costs by payer type and stay type, infection rates, and patient satisfaction indicator of the health dashboard according to the invention;

FIG. 3 is an example of a dashboard screen that highlights bed occupancy rates, patient turnover, efficiency and costs, follow-up rates, and waiting times by division of the health dashboard according to the invention; and

FIG. 4 is an example of a dashboard graph of a prediction of a future condition of a display attribute of the health dashboard according to the invention.

### DETAILED DESCRIPTION OF THE INVENTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Throughout this specification, the word “comprise,” or variations thereof such as “comprises” or “comprising,” will be understood to imply the inclusion of a stated element, integer or step, or group of elements integers or steps, but not the exclusion of any other element, integer or step, or group of elements, integers or steps.

Throughout this specification, the phrase “health dashboard,” or variations thereof such as “dashboard” or “healthcare dashboard,” will be understood to mean an interactive visual tool that consolidates and displays key metrics related to healthcare performance and patient care. It presents data through charts, graphs, and tables to make complex information easily understandable and accessible. Health dashboards enable real-time monitoring, performance tracking, and informed decision-making by highlighting critical indicators such as patient outcomes, treatment costs, and resource utilization.

Statistical data and visualizations on several metrics can be shown on the dashboard such as:
Length of Stay: Average length of stay in days across different quarters.
Admissions & Readmission Rate: Number of admissions and readmissions, along with the average 30-day readmission rate.
Age Groups: Distribution of patients by age groups.
Treatment Costs: Average treatment costs broken down by age groups and payer types.
Nurse-Patient Ratios: Ratios in various units such as nursery, trauma, emergency rooms, surgical rooms, and rehabilitation units, with differentiation between day and night shifts.
Hospital-Acquired Infections: Percentage of cases with surgical site infections and central-line catheter infections.
Occupancy: Current bed occupancy rate, number of patients, and number of beds.
Patient Turnover: Forecasted patient turnover and turnover rate by the end of the month.
Patient Satisfaction: Satisfaction scores and their change from the previous month.
Efficiency & Costs: Average total cost per patient, staff-to-patient ratio, room turnover time, and drug cost per patient by department.
Appointments: Follow-up rate, cancellation and missed appointment rates by department.
Patient Numbers: Total patients admitted and treated, both inpatient and outpatient, by different medical divisions.
Waiting Times: Average waiting times by division.

### INDEX OF LABELLED FEATURES IN FIGURES

Features are listed in numeric order by Figure in numeric order.

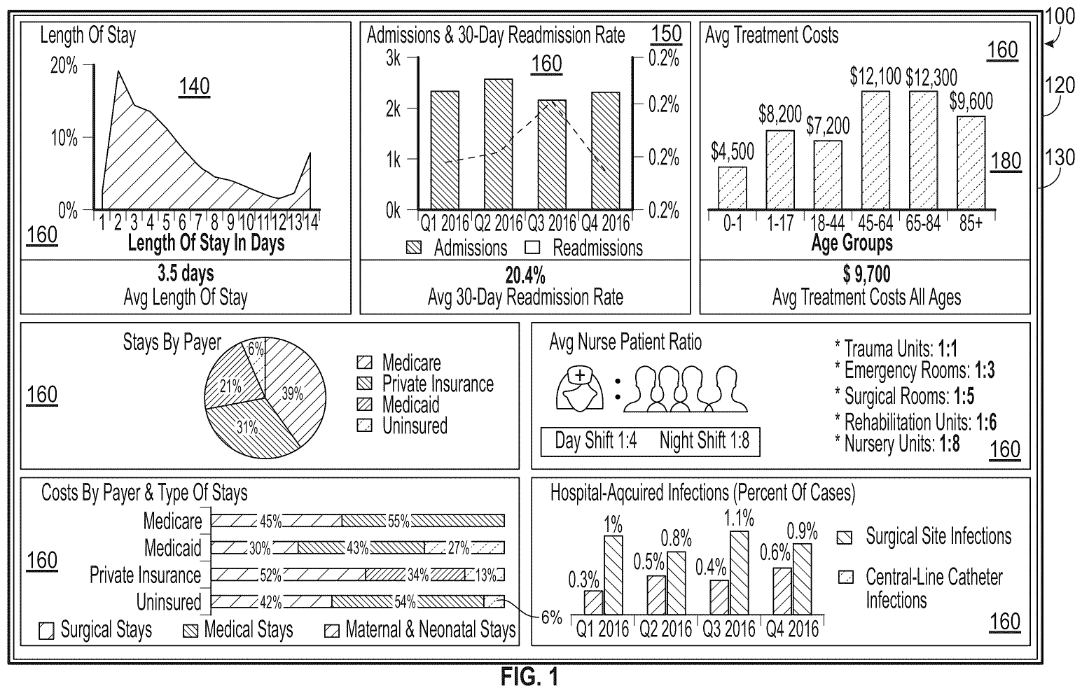

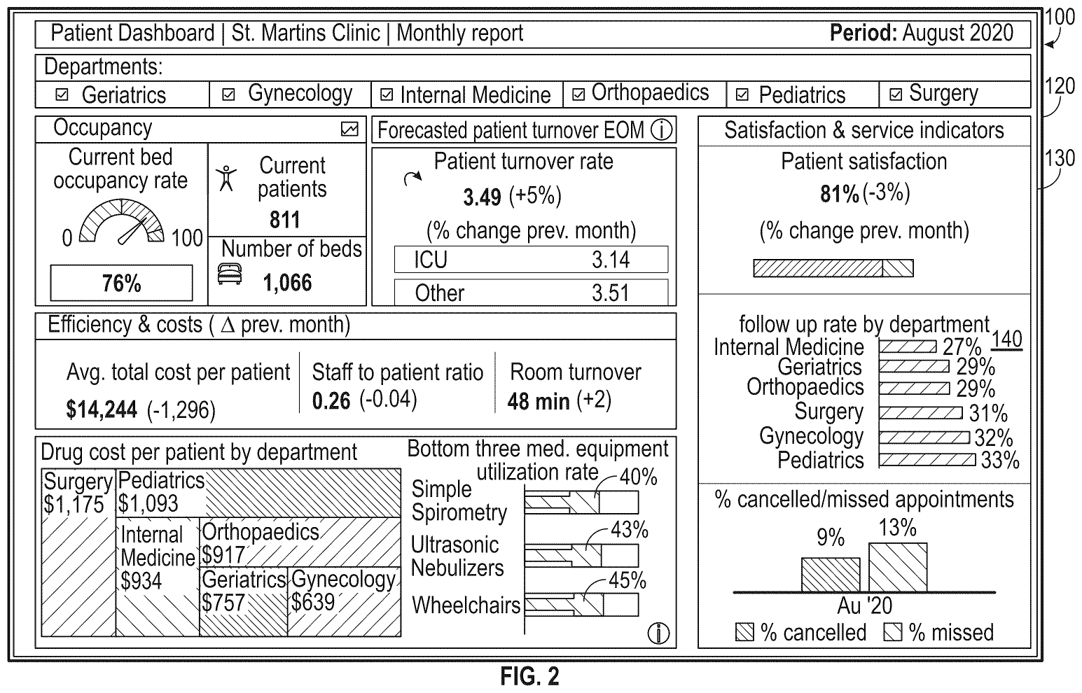

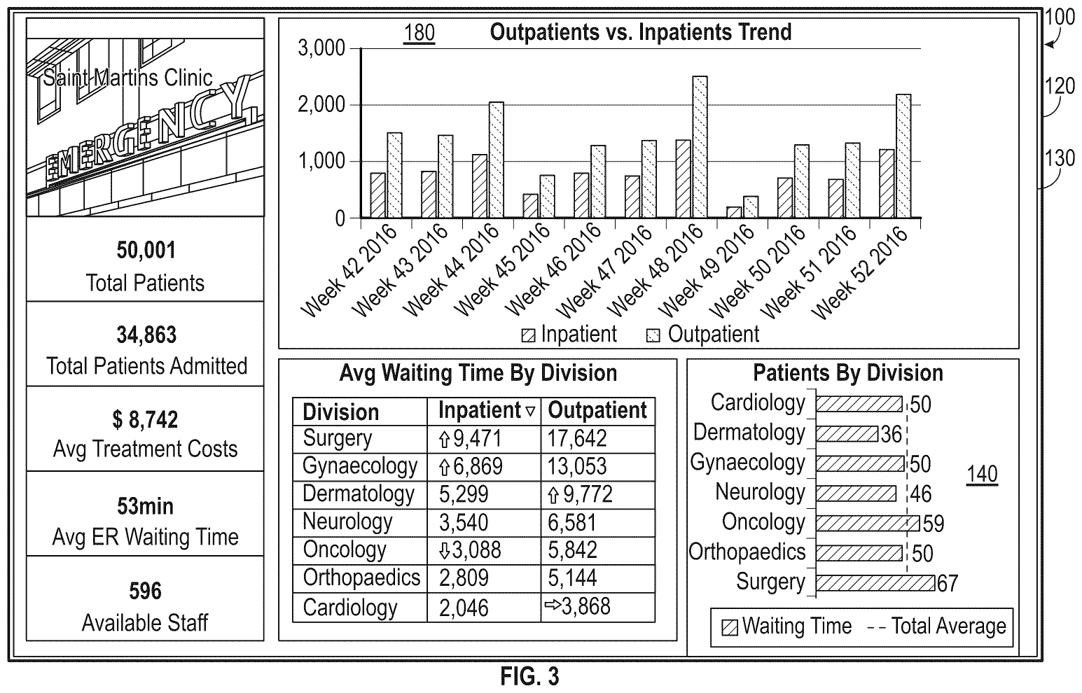

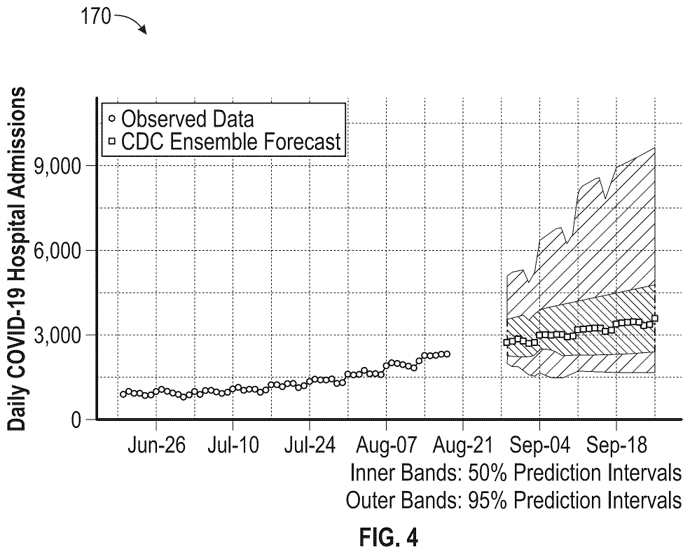

Referring to the Figures, there is shown in FIGS. 1, 2, 3, and 4 the following features:
Element 100 which is a health dashboard.
Element 120 which is a display window.
Element 130 which is a panel.
Element 140 which is a data visualization.
Element 150 which is a historical chart.
Element 160 which is a tile.
Element 170 which is a prediction of a future condition of a display attribute.
Element 180 which is a bar chart.

Elements not shown in the figures include: a health information record, a health condition, a disease condition, a processor, and a memory running an algorithm.

### Example of how to Make the Present Invention

### 1. System Architecture

### Hardware:

Processor: A capable processor, such as INTEL i7 processor or AMD RYZEN processor, to handle multiple concurrent data processing and visualization tasks.
Memory: Sufficient memory (at least 16 GB of RAM) to store and process user data efficiently.

### Software:

Operating System: An OS that supports necessary software tools and libraries, such as LINUX operating system, WINDOWS operating system, or macOS operating system.
Programming Languages: PYTHON programming language for backend processing, JAVASCRIPT programming language for frontend interactions.

### Frameworks/Libraries:

Backend: FLASK framework or DJANGO framework for web application development.
Frontend: REACT library, ANGULAR framework, or VUE.JS framework for building dynamic user interfaces.
Data Visualization: D3.js, PLOTLY data analytics and visualization tool, or Chart.js for creating interactive charts and graphs.

### 2. Database Design

### Attributes:

User ID: A unique identifier for each user.
Name: Full name of the user.
Age: Age of the user.
Gender: Gender of the user.
Weight: Current weight of the user.
Height: Current height of the user.
Blood Pressure: Recorded blood pressure readings.
Heart Rate: Recorded heart rate readings.
Blood Sugar Level: Recorded blood sugar levels.
Cholesterol Level: Recorded cholesterol levels.
Medication: List of current medications.
Allergies: Known allergies.
Health Conditions: Diagnosed health conditions.
Disease Conditions: Any specific diseases diagnosed.
History of Medical Visits: Records of past medical visits, including dates and reasons for visits.

### 3. Backend Implementation

Data Collection: Implement APIs for users to submit their health information. Use secure forms for data entry, ensuring data privacy and compliance with regulations such as HIPAA.
Data Storage: Utilize a relational database (e.g., POSTGRESQL relational database management system, MYSQL relational database management system) for structured data storage. Consider a NoSQL database (e.g., MONGODB database) for flexible, schema-less data storage.
Data Processing: Develop algorithms to process and analyze the collected data. Implement data validation and cleaning processes to ensure accuracy. Use statistical methods and machine learning models for health trend analysis and predictions.

### 4. Frontend Implementation

User Interface (UI): Design a clean and intuitive UI using HTML, CSS, and JAVASCRIPT programming language. Ensure the UI is user-friendly and accessible, following best practices for UX/UI design.
Tiles Design: Divide the dashboard into six tiles, each designed to display different types of visualizations. Ensure that each tile is customizable and can be rearranged by the user as needed.

### 5. Data Visualization Tiles:

First Data Visualization: A pie chart showing the distribution of different health conditions (e.g., percentage of users with hypertension, diabetes, etc.).
Second Data Visualization: A line chart showing trends in blood pressure readings over time, allowing users to track their blood pressure history.
Third Data Visualization: A scatter plot displaying the relationship between weight and heart rate, helping users understand how their weight might impact their cardiovascular health.
First Bar Chart: A bar chart displaying the frequency of medication intake, providing insights into medication adherence.
Second Bar Chart: A bar chart showing the number of medical visits over the past year, highlighting patterns in healthcare utilization.
Historical Chart: A line or area chart depicting the historical trend of blood sugar levels, helping users monitor their glucose management over time.

### 6. Common Y-Axis Configuration

Ensure that all tiles use a common y-axis for uniformity in data representation. This approach facilitates easier comparison and analysis of trends across different health attributes. For example, if multiple tiles display data over time, aligning the y-axes allows users to see how different health metrics change in relation to each other.

### 7. Additional Features

### Interactive Elements:

Tooltips: Allow users to hover over data points to see detailed information.
Zoom and Pan: Enable users to zoom in on specific sections of the charts and pan across data ranges.
Drill-downs: Provide the ability to click on chart elements to view more detailed data.

### Filter Options:

Date Range: Allow users to filter data based on specific date ranges.
Conditions: Enable filtering based on specific health conditions or diseases.
Customizable Views: Allow users to customize which attributes they want to view on the dashboard.
Responsive Design: Ensure the dashboard is responsive and accessible on various devices, including desktops, tablets, and smartphones. Implement adaptive layouts that adjust the display based on the screen size.

By following this detailed approach, you can create a comprehensive and visually appealing health dashboard. The dashboard will allow users to monitor and analyze their health data effectively, providing valuable insights into their health trends and conditions.

In a preferred embodiment of the invention, there is a user health dashboard for collecting and displaying a health information record of a user, a health condition of said user, and/or a disease condition having at least fifteen attributes of said user comprising a processor having a memory running an algorithm configured to present to said user a display window configured to show fifteen attributes of said health information record in up to six tiles having a common y-axis for a data display on a panel wherein said tiles are selected from a first data visualization configured to display at least one attribute of said health information record, a second data visualization configured to display at least one attribute of said health information record, a third data visualization configured to display at least one attribute of said health information record, a first bar chart configured to display at least one attribute of said health information record, a second bar chart configured to display at least one attribute of said health information record, and a historical chart configured to display at least one attribute of said health information record wherein all tiles use a common data element of an attribute such as a collection of entries or readings over time.

In an alternate embodiment of the invention, there is a user health dashboard wherein at least one tile comprises a prediction of a future condition of a display attribute.

In an alternate embodiment of the invention, there is a user health dashboard wherein said prediction of said future condition is derived from said algorithm and/or an artificial intelligence algorithm.

In an alternate embodiment of the invention, there is a user health dashboard having not less than three tiles consisting of said first data visualization, said first bar chart, and said historical chart.

In an alternate embodiment of the invention, there is a user health dashboard further configured for said health condition comprising a display of tiles for attributes related to said health condition.

In an alternate embodiment of the invention, there is a user health dashboard further configured for said disease condition comprising a display of tiles for attributes related to said disease condition.

In a preferred embodiment of the invention, there is a user health dashboard for collecting and displaying a health condition of a user having at least fifteen attributes of said user comprising a processor having a memory running an algorithm configured to present to said user a display window configured to show fifteen attributes of said health condition in up to six tiles having a common y-axis for a data display on a panel wherein said tiles are selected from a first data visualization configured to display at least one attribute of said health condition, a second data visualization configured to display at least one attribute of said health condition, a third data visualization configured to display at least one attribute of said health condition, a first bar chart configured to display at least one attribute of said health condition, a second bar chart configured to display at least one attribute of said health condition, and a historical chart configured to display at least one attribute of said health condition wherein all tiles use a common data element of an attribute such as a collection of entries or readings over time.

In an alternate embodiment of the invention, there is a user health dashboard wherein a tile comprises a prediction of a future condition of a display attribute.

In an alternate embodiment of the invention, there is a user health dashboard wherein said prediction of said future condition is derived from said algorithm and/or an artificial intelligence algorithm.

In an alternate embodiment of the invention, there is a user health dashboard having not less than three tiles consisting of said first data visualization, said first bar chart, and said historical chart.

In a preferred embodiment of the invention, there is a user health dashboard for collecting and displaying a disease condition of a user having at least fifteen attributes of said user comprising a processor having a memory running an algorithm configured to present to said user a display window configured to show fifteen attributes of said disease condition in up to six tiles having a y-axis for a data display on a panel wherein said tiles are selected from a first data visualization configured to display at least one attribute of said disease condition, a second data visualization configured to display at least one attribute of said disease condition, a third data visualization configured to display at least one attribute of said disease condition, a first bar chart configured to display at least one attribute of said disease condition, a second bar chart configured to display at least one attribute of said disease condition, and a historical chart configured to display at least one attribute of said disease condition wherein all tiles use a common data element of an attribute such as a collection of entries or readings over time.

In an alternate embodiment of the invention, there is a user health dashboard wherein a tile comprises a prediction of a future condition of a display attribute.

In an alternate embodiment of the invention, there is a user health dashboard wherein said prediction of said future condition is derived from said algorithm and/or an artificial intelligence algorithm.

In an alternate embodiment of the invention, there is a user health dashboard having not less than three tiles consisting of said first data visualization, said first bar chart, and said historical chart.

The health dashboard of the present invention allows the public to better understand the healthcare data as presented in the dashboard, the provider to efficiently create dashboards without having to spend time and resources to develop an effective dashboard, AI to be incorporated into a healthcare dashboard, predictive analytics to be incorporated into a healthcare dashboard, the provider to display a healthcare dashboard that can be effectively presented on various devices, the public to receive the right amount of healthcare data to make an immediate healthcare decision, the public no matter their age, educational level, etc. to effectively receive and understand healthcare data, and the right healthcare data to be presented for improving public healthcare outcomes.

Some key benefits and functions of a health dashboard are as follows:
Visual Representation: Uses charts, graphs, tables, and other visual elements to make complex data easily understandable.
Key Metrics: Highlights critical performance indicators such as patient outcomes, treatment costs, admission and readmission rates, staff-to-patient ratios, occupancy rates, and more.
Real-Time Data: Often updated in real-time or near-real-time to provide the most current information for decision-making.
Interactivity: Allows users to drill down into specific data points, filter information, and customize views to analyze particular aspects of healthcare performance.
Performance Monitoring: Helps healthcare administrators and professionals track performance against benchmarks and goals, identify trends, and pinpoint areas needing improvement.
Decision Support: Facilitates informed decision-making by providing a comprehensive overview of hospital operations, resource utilization, patient care quality, and financial performance.
Transparency and Accountability: Promotes transparency in healthcare operations and holds departments accountable for their performance.

A user health dashboard is significantly more than just software because it is a sophisticated system designed to collect, process, and display a comprehensive health information record of a user. A user health dashboard is a sophisticated system designed to collect and display a comprehensive health information record of a user, encompassing their health condition and/or disease condition with at least fifteen attributes. This dashboard features a processor with memory running an algorithm that presents a display window to the user. This window is configured to show the fifteen attributes of the health information record in up to six tiles, all sharing a common y-axis for consistent data display on a panel. These tiles can be chosen from a variety of data visualizations and charts, each designed to display one or more attributes of the health information record.

The tiles available for selection include: First Data Visualization: Displays at least one attribute of the health information record. Second Data Visualization: Displays at least one attribute of the health information record. Third Data Visualization: Displays at least one attribute of the health information record. First Bar Chart: Configured to display at least one attribute of the health information record. Second Bar Chart: Configured to display at least one attribute of the health information record. Historical Chart: Displays at least one attribute of the health information record, focusing on a collection of entries or readings over time.

The use of a common y-axis across all tiles ensures a unified and easily interpretable data presentation. The dashboard is designed to handle and visualize a significant amount of data, making it much more than just a basic software application. It provides a detailed and interactive interface for users to monitor their health status comprehensively.

This dashboard is implemented on a processor with a memory running a specialized algorithm that presents the data to the user in a clear and organized manner.

### Key Features and Attributes:

Processor and Memory: The system is powered by a processor with an adequate memory capacity to run complex algorithms.

Algorithm: The algorithm is tailored to manage, analyze, and display health data efficiently.

Display Window: The user interface includes a display window where information is presented.

### Data Visualization and Tile Configuration:

The dashboard is designed to show at least fifteen attributes of the user's health information record. These attributes are displayed across up to six tiles, each sharing a common y-axis for data consistency and comparison. The tiles can display various forms of data visualization, including:

First Data Visualization: This could be a line graph, pie chart, or any other visual representation showing at least one attribute of the user's health data.

Second Data Visualization: Similar to the first, this tile displays another attribute using a different or similar type of visualization.

Third Data Visualization: Another visual representation focusing on a different attribute of the health record.

First Bar Chart: A bar chart that highlights one or more attributes of the user's health data.

Second Bar Chart: Another bar chart for different attributes, allowing for comparative analysis.

Historical Chart: A chart that shows historical data trends, helping the user to see changes and patterns over time.

### Common Data Elements:

Each tile uses a common data element for consistency and easier interpretation. This could include a collection of entries or readings over a specified period. For example, if tracking blood pressure, each tile might show systolic and diastolic readings, trends over time, comparisons with normal ranges, and historical data.

### Application and Use Case:

The health dashboard goes beyond being a simple software application by offering:

Comprehensive Data Collection: It gathers diverse health metrics and organizes them systematically.

Real-Time Processing: It processes and updates data in real-time, providing up-to-date information.

User-Friendly Interface: The display is designed to be intuitive, allowing users to easily understand and interact with their health data.

Analytical Insights: The system provides insights and trends, helping users make informed health decisions.

In essence, the user health dashboard is a powerful tool that leverages advanced algorithms and data visualization techniques to provide a holistic view of an individual's health status, making it a valuable asset for personal health management.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1-14. (canceled)

15. A user health dashboard for collecting and displaying a health information record of a user, a health condition of said user, or a disease condition, said health information record having at least fifteen attributes of said user, comprising:
a processor having a memory running an algorithm configured to present to said user, in a single display window, said at least fifteen attributes of said health information record in up to six tiles simultaneously visible in said single display window,
wherein all tiles display respective visualizations of a same attribute type measured over time using a synchronized common y-axis scale conditioned wherein said synchronized common y-axis scale will adjust all of said tiles automatically when any one tile is zoomed or paned,
wherein said tiles are selected from:
a first data visualization configured to display at least one attribute of said health information record,
a second data visualization configured to display at least one attribute of said health information record,
a third data visualization configured to display at least one attribute of said health information record,
a first bar chart configured to display at least one attribute of said health information record,
a second bar chart configured to display at least one attribute of said health information record, and
a historical chart configured to display at least one attribute of said health information record,
wherein all tiles use a common data element comprising a collection of entries or readings for one of said at least fifteen attributes type over time.

16. The user health dashboard of claim 15, wherein at least one tile further comprises a graphical prediction of a future condition of a displayed attribute, said graphical prediction being graphically integrated into said tile alongside historical values for said displayed attribute to form a continuous time-series display.

17. The user health dashboard of claim 16, wherein said graphical prediction of said future condition is derived from a neural network that retrains itself when new health data comes in and updates predictive weights in real time, said neural network being adaptive to newly collected data.

18. The user health dashboard of claim 15, having not less than three tiles consisting of said first data visualization, said first bar chart, and said historical chart all displayed concurrently within said single display window and using said synchronized common y-axis scale.

19. The user health dashboard of claim 15, further configured for said health condition by displaying said tiles in an adjacent arrangement wherein said tiles are side-by-side with aligned time axes so said tiles can be compared point-by-point over a same time window to facilitate condition-specific comparative analysis.

20. The user health dashboard of claim 15, further configured for said disease condition by displaying said tiles in an adjacent arrangement to facilitate a disease-specific comparative analysis, wherein each tile includes both current and predicted future values of said attributes related to said disease condition.

21. A user health dashboard for collecting and displaying a health condition of a user, said health condition having at least fifteen attributes of said user, comprising:
a processor having a memory running an algorithm configured to present to said user, in a single display window, said at least fifteen attributes of said health condition in up to six tiles simultaneously visible in said single display window,
wherein all of said tiles display respective visualizations of different aspects of a same health condition measured over time using a synchronized common y-axis scale,
wherein said tiles are selected from:
a first data visualization configured to display at least one attribute of said health condition,
a second data visualization configured to display at least one attribute of said health condition,
a third data visualization configured to display at least one attribute of said health condition,
a first bar chart configured to display at least one attribute of said health condition,
a second bar chart configured to display at least one attribute of said health condition, and
a historical chart configured to display at least one attribute of said health condition,
wherein all tiles use a common data element comprising a collection of entries or readings for an attribute type over time.

22. The user health dashboard of claim 21, wherein at least one tile further comprises a graphical prediction of a future condition of a displayed attribute, said graphical prediction being graphically integrated into said tile alongside historical values for said displayed attribute to form a continuous time-series display.

23. The user health dashboard of claim 22, wherein said graphical prediction of said future condition is derived from a trained artificial intelligence model configured to forecast a plurality of future values of said at least fifteen attributes by learning from historical data trends, said trained artificial intelligence model being adaptive to newly collected data.

24. The user health dashboard of claim 21, having not less than three tiles consisting of said first data visualization, said first bar chart, and said historical chart, all displayed concurrently within said single display window and using said synchronized common y-axis scale.

25. A user health dashboard for collecting and displaying a disease condition of a user, said disease condition having at least fifteen attributes of said user, comprising:
a processor having a memory running an algorithm configured to present to said user, in a single display window, said at least fifteen attributes of said disease condition in up to six tiles simultaneously visible in said single display window,
wherein all of said tiles display respective visualizations of different aspects of a same disease condition measured over time using a synchronized common y-axis scale,
wherein said tiles are selected from:
a first data visualization configured to display at least one attribute of said disease condition,
a second data visualization configured to display at least one attribute of said disease condition,
a third data visualization configured to display at least one attribute of said disease condition,
a first bar chart configured to display at least one attribute of said disease condition,
a second bar chart configured to display at least one attribute of said disease condition, and
a historical chart configured to display at least one attribute of said disease condition,
wherein all tiles use a common data element comprising a collection of entries or readings for an attribute type over time.

26. The user health dashboard of claim 25, wherein at least one tile further comprises a graphical prediction of a future condition of a displayed attribute, said graphical prediction being graphically integrated into said tile alongside historical values for said displayed attribute to form a continuous time-series display.

27. The user health dashboard of claim 26, wherein said prediction of said future condition is derived from a trained artificial intelligence model configured to forecast a plurality of future values of said displayed attribute by learning from historical data trends, said trained artificial intelligence model being adaptive to newly collected data.

28. The user health dashboard of claim 25, having not less than three tiles consisting of said first data visualization, said first bar chart, and said historical chart, all displayed concurrently within said single display window and using said synchronized common y-axis scale.
